(19) United States
(12) Patent Application Publication
(10) Pub. No.: US 2001/0018402 A1
(43) Pub. Date: Aug. 30, 2001

(54) Fixed bed raney copper catalyst

(76) Inventors: Daniel Ostgard, Kleinostheim (DE); Monika Berweiler, Maintal (DE); Karsten Seelbach, Haltern-Hullern (DE)

Correspondence Address: Smith, Gambrell & Russell, LLP, Beveridge, DeGrandi, Weilacher & Young, Intellectual Property Group, 1850 M Street, N.W., Suite 800, Washington, DC, 20036, US

(21) Appl. No.: 09/778,804

(22) Filed: Feb. 8, 2001

(30) Foreign Application Priority Data
Feb. 18, 2000 (DE) 00103547.6

(51) Int. Cl. B01J 23/70; B01J 23/72; B01J 23/00; C07C 51/16

(52) U.S. Cl. 502/345; 502/318; 502/331; 502/316; 562/526

(57) ABSTRACT

A fixed bed Raney copper catalyst, which is doped with iron, noble metals or other metals, is employed as the fixed bed catalyst in the fixed bed dehydrogenation of alcohols.

### INTRODUCTION AND BACKGROUND

The present invention relates to a fixed bed Raney copper catalyst, a process for its preparation and a process for the dehydrogenation of alcohols.

It is known to dehydrogenate diethanolamine to give iminodiacetic acid. (U.S. Pat. No. 5,689,000; WO 96/01146; WO 92/06949; JP-OS 091 55 195; U.S. Pat. No. 5,292,936; U.S. Pat. No. 5,367,112; CA 212 10 20).

### SUMMARY OF THE INVENTION

The present invention provides a fixed bed Raney copper catalyst which is prepared as tablets, extrudates, hollow bodies, fiber tablets, granules bonded to a support and disc-shaped granules. The fixed bed Raney catalyst can be doped by means of metal from the group consisting of iron and/or noble metal. It can optionally comprise other doping metals, e.g. Bi, Sn, Sb, Pb, Ge, Cr, Mo, Ti, Ni, Ta, Zr, V, Mn, W, Co and/or Nb and/or mixtures thereof.

The doping metal can be both alloyed into the copper and subsequently coated on.

The Raney copper according to the invention can comprise the doping elements in an amount of 10 ppm to 1 wt. %. The noble metal doping can be 10 to 50,000 ppm, preferably 500 to 50,000 ppm. The doping metals can be chosen from the group consisting of iron and palladium, platinum, gold, silver, iridium, ruthenium and/or rhodium.

In particular, a metal from the group consisting of Pt, Pd and/or Fe can be chosen for the doping.

The average particle size of the fixed bed Raney copper catalyst according to the invention can be from 0.05 mm to 20 mm.

The average particle size of the fixed bed Raney copper catalyst according to the invention is of importance for the use in oxidation reactions or dehydrogenation reactions of alcohols.

The fixed bed Raney copper catalyst according to the invention is advantageously not deactivated by an undesirable poisoning or an undesirable abrasion.

The invention also provides a process for the preparation of the fixed bed Raney copper catalyst according to the invention, which comprises preparing a fixed bed Raney catalyst by the known route, shaping it, activating it, doping it with at least one doping metal, washing it and drying it.

The doping by means of a doping metal can be carried out by introducing the activated catalyst into a column reactor with a solution circulation and adding the doping metal solution to the circulating solution.

The shaping of the catalyst can be carried out by the known route.

In a particular embodiment, the catalyst doped according to the invention can be shaped into hollow spheres. For this, the alloy powder can be suspended in a aqueous solution with optionally further constituents and this suspension can be sprayed on to readily combustible beads, for example polystyrene beads. This coating operation can optionally be repeated. After the coating, the beads can in each case be dried in a stream of air.

The readily combustible beads are then burned out. The resulting hollow spheres are then activated by means of sodium hydroxide solution and doped by means of metal salt solution, washed and dried.

The invention also provides a process for the catalytic dehydrogenation of alcohols, which comprises using as a fixed bed catalyst a fixed bed Raney copper catalyst doped with iron and/or noble metal, and optionally other suitable doping metals.

### DETAILED DESCRIPTION OF INVENTION

The process according to the invention for the dehydrogenation of alcohols can be used for the dehydrogenation of glycols and/or amino-alcohols. The fixed bed catalyst can be employed here as tablets, extrudates, hollow bodies, fibre tablets, granules bonded to a support and disc-shaped granules.

The alcohols which can be dehydrogenated according to the invention can be mono- or polyhydric alcohols. They can be aliphatic, cyclic or aromatic compounds, including polyether glycols, which react with a strong base to give the carboxylates.

It is necessary here that the alcohol and the resulting carboxylate are stable in strongly basic solution and the alcohol is at least somewhat soluble in water.

Suitable primary monohydric alcohols can include:

aliphatic alcohols, which can be branched, straight-chain, cyclic or aromatic alcohols, such as, for example, benzyl alcohol, it being possible for these alcohols to be substituted by various groups which are stable to bases.

Suitable aliphatic alcohols can be ethanol, propanol, butanol, pentanol or the like.

According to the invention, glycols can be oxidized to carboxylic acids or dehydrogenated.

Thus, for example, ethylene glycol can be dehydrogenated to glycollic acid (monocarboxylic acid) and the dicarboxylic acid oxalic acid can be prepared by subsequent reaction with KOH.

Amino-alcohols can also be dehydrogenated with the Raney copper doped according to the invention with noble metal, to give the corresponding aminocarboxylic acids. The amino-alcohols can contain 1 to 50 C atoms.

Thus, for example, N-methylethanolamine can be dehydrogenated to sarcosine; THEEDA to EDTA; monoethanolamine to glycine; diethanolamine to iminodiacetic acid; 3-amino-1-propanol to beta-alanine; 2-amino-1-butanol to 2-aminobutyric acid.

In one embodiment of the invention, alcohols of the formula

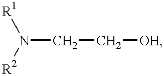

in which R¹ and R² in each case denote hydrogen; hydroxyethyl; —CH₂CO₂H; an alkyl group having 1 to 18 C atoms; an aminoalkyl group having 1 to 3 C atoms; a hydroxyalkylaminoalkyl group having 2 to 3 C atoms and phosphonomethyl, can be dehydrogenated by the process according to the invention.

The amino-alcohols which can be employed according to the invention are known. If R¹ and R² are hydrogen, the amino-alcohol is diethanolamine.

If R¹ and R² are hydroxyethyl, the amino-alcohol is triethanolamine. The resulting aminocarboxylic acid salts of these starting amino-alcohols should be the salts of glycine, iminodiacetic acid or nitrilotriacetic acid. Further amino-alcohols include N-methylethanolamine, N,N-dimethylethanolamine, N-ethylethanolamine, N-isopropylethanolamine, N-butylethanolamine, N-nonylethanolamines, N-(2-aminoethyl)ethanolamine, N-(3-aminopropyl)ethanolamine, N,N-diethylethanolamine, N,N-dibutylethanolamine, N-methyldiethanolamine, N-ethyldiethanolamine, N-isopropyldiethanolamine, N-butyldiethanolamine, N-ethyl,N-(2-aminoethyl)-ethanolamine, N-methyl,N-(3-aminopropyl)ethanolamine, tetra(2-hydroxyethyl)ethylenediamine, and the like.

Further examples of aminocarboxylic acid salts are the salts of N-methylglycine, N,N-dimethylglycine, N-ethylglycine, N-isopropylglycine, N-butylglycine, N-nonylglycine, N-(2-aminoethyl)glycine, N-3-aminopropyl)glycine, N,N-diethylglycine, N,N-dibutylglycine, N-methyliminodiacetic acid, N-ethyliminodiacetic acid, N-isopropyliminodiacetic acid, N-butyliminodiacetic acid, N-ethyl, N-(2-aminoethyl)glycine, N-methyl-N-(3-aminopropyl)glycine, ethylenediaminetetraacetic acid, and so on.

R¹ or R² can also be a phosphonomethyl group, where the starting amino compound can be N-phosphonomethylethanolamine and the resulting amino acid can be N-phosphonomethylglycine. If of R¹ or R² one R=phosphonomethyl and the other R=—CH₂CH₂OH, the resulting amino acid would be N-phosphonomethyliminodiacetic acid, which can be converted into N-phosphonomethylglycine by the known route. If of R¹ or R² one R=phosphonomethyl and the other R=an alkyl group, the resulting acid would be N-alkyl-N-phosphonomethylglycine, which can be converted further into N-phosphonomethylglycines in accordance with U.S. Pat. No. 5,068,404.

The process according to the invention can be carried out at a temperature of 50 to 250° C., preferably 80 to 200° C., under a pressure of 0.1 to 200 bar, preferably normal pressure to 50 bar.

Pressure is necessary because the alcohols have a high vapour pressure. When the hydrogen is let off, the alcohol would also be let off under too low a pressure.

The process according to the invention has the following advantages:

Known pulverized catalysts have the disadvantage that they can be used only in a discontinuous process and must be separated off from the reaction medium by expensive settling and/or filtration after the catalytic reaction.

The fixed bed catalysts according to the invention are suitable for continuous processes. The reaction solution can be separated from the catalyst more easily.

The stabilized catalysts and catalysts with no non-activated alloy also have an advantage in the more basic solution required, which must be used for the alcohol dehydrogenation. These catalysts are not activated further during the reaction. The stabilization of the catalysts could either be carried out with a higher content of Cu binder, in which case the copper content can be 2.5 to 70%, or with a higher calcining temperature, but without the formation of alpha-aluminium oxide.

The noble metals, iron or fixed bed Raney copper catalysts doped with other metals furthermore have the advantage that they have an improved resistance to chemical or mechanical deactivation. Examples of chemical deactivation could be poisonous compounds in the educt, poisonous by-products and decomposed compounds on the catalytic surface.

Examples of mechanical deactivation could be abrasion or disintegration of the shaped bodies.

### EXAMPLE 1 (COMPARISON EXAMPLE)

In accordance with EP 0 6 48 534 A1, for a comparison catalyst which comprises 1,000 g alloy powder of 50% Cu and 50% Al, 100 g pure copper powder (99% copper, d50=21 μμm) and 25 g ethylene bis-stearoylamide, a free-flowing catalyst mixture which can be pelletted is prepared with the addition of about 150 g water. Tablets with a diameter of 3 mm and a thickness of 3 mm are pressed from this mixture. The shaped bodies are calcined at 700° C. for 2 hours. The tablets are activated in 20% sodium hydroxide solution at 40-80° C. for 2 hours after the calcining. Under the conditions of the use example, this catalyst needs more than 7 hours for the dehydrogenation of 378.0 g diethanolamine to iminodiacetic acid.

### EXAMPLE 2 (COMPARISON EXAMPLE)

In accordance with EP 0 6 48 534 A1, for a comparison catalyst which comprises 1,000 g alloy powder of 50% Cu and 50% Al, 675 g pure copper powder (99% copper, d50=21 μμm) and 25 g ethylene bis-stearoylamide, a free-flowing catalyst mixture which can be pelletted is prepared with the addition of about 150 g water. Tablets with a diameter of 3 mm and a thickness of 3 mm are pressed from this mixture. The shaped bodies are calcined at 700° C. for 2 hours. The tablets are activated in 20% sodium hydroxide solution at 40-80° C. for 2 hours after the calcining. Under the conditions of the use example, for the dehydrogenation of 189.0 g diethanolamine to iminodiacetic acid this catalyst needs 130 minutes for the first cycle and 150 minutes for cycles 2, 3 and 4.

### EXAMPLE 3

In accordance with EP 0 6 48 534 A1, for a catalyst which comprises 1,000 g alloy powder of 50% Cu and 50% Al, 100 g pure copper powder (99% copper, d50=21 μμm) and 25 g ethylene bis-stearoylamide, a free-flowing catalyst mixture which can be pelletted is prepared with the addition of about 150 g water. Tablets with a diameter of 3 mm and a thickness of 3 mm are pressed from this mixture. The shaped bodies are calcined at 700° C. for 2 hours. The tablets are activated in 20% sodium hydroxide solution at 40-80° C. for 2 hours after the calcining. Hexachloroplatinum is then added to the suspension of the washed catalyst. The pH is adjusted and the suspension is stirred further. The doped catalyst is then washed. The platinum content of the catalyst is 1%.

### EXAMPLE 4

In accordance with EP 0 6 48 534 A1, for a catalyst which comprises 1,000 g alloy powder of 50% Cu and 50% Al, 675 g pure copper powder (99% copper, d50=21 μμm) and 25 g ethylenebis-stearoylamide, a free-flowing catalyst mixture which can be pelletted is prepared with the addition of about 150 g water. Tablets with a diameter of 3 mm and a thickness of 3 mm are pressed from this mixture. The shaped bodies are calcined at 700° C. for 2 hours. The tablets are activated in 20% sodium hydroxide solution at 40-80° C. for 2 hours after the calcining. Hexachloroplatinum is then added to the suspension of the washed catalyst. The pH is adjusted and the suspension is stirred further. The doped catalyst is then washed. The platinum content of the catalyst is 1%.

### EXAMPLE 5

In accordance with EP 0 6 48 534 A1, for a catalyst which comprises 1,000 g alloy powder of 50% Cu and 50% Al, 100 g pure copper powder (99% copper, d50=21 μμm) and 25 g ethylene bis-stearoylamide, a free-flowing catalyst mixture which can be pelletted is prepared with the addition of about 150 g water. Tablets with a diameter of 3 mm and a thickness of 3 mm are pressed from this mixture. The shaped bodies are calcined at 700° C. for 2 hours. The tablets are activated in 20% sodium hydroxide solution at 40-80° C. for 2 hours after the calcining. Iron(III) chloride is then added to the suspension of the washed catalyst. The pH is adjusted and the suspension is stirred further. The doped catalyst is then washed. The iron content of the catalyst is 3%.

### EXAMPLE 6

In accordance with EP 0 6 48 534 A1, for a catalyst which comprises 1,000 g alloy powder of 50% Cu and 50% Al, 675 g pure copper powder (99% copper, d50=21 μμm) and 25 g ethylene bis-stearoylamide, a free-flowing catalyst mixture which can be pelletted is prepared with the addition of about 150 g water. Tablets with a diameter of 3 mm and a thickness of 3 mm are pressed from this mixture. The shaped bodies are calcined at 700° C. for 2 hours. The tablets are activated in 20% sodium hydroxide solution at 40-80° C. for 2 hours after the calcining. Iron(III) chloride is then added to the suspension of the washed catalyst. The pH is adjusted and the suspension is stirred further. The doped catalyst is then washed. The iron content of the catalyst is 3%.

### EXAMPLE 7

A coating solution is prepared by suspending 800 g of an alloy of 50% Cu/50% Al and 104 g copper powder in 1,000 ml aqueous solution with a content of 5 wt. % polyvinyl alcohol and 1.25 wt. % glycerol. This suspension is then sprayed on to 2,000 ml polystyrene beads in the range from 4 to 5 mm, while these are suspended in upwards-flowing air. After the polystyrene beads have been coated with the abovementioned solution, the beads are dried in upwards-flowing air at temperatures of up to 80° C. (Higher temperatures can also be used). These dried, coated polystyrene beads have a bulk density of 0.26 g/ml, and half of these beads are coated further with an alloy solution.

The solution for the second layer comprises 800 g of an alloy of 50% Cu/50% Al and 104 g copper powder suspended in 1,000 ml aqueous solution with a content of 5 wt. % polyvinyl alcohol and 1.25 wt. % glycerol. This suspension is then sprayed on to 1,000 ml of the abovementioned polystyrene beads which have been precoated with Cu/Al and dried, while these are suspended in an upwards-directed stream of air.

After the polystyrene beads have been coated with the abovementioned solution, the beads are dried in upwards-flowing air at temperatures of up to 80° C. Higher temperatures can also be used. The dried, coated beads are then heated at 550° C. in a controlled stream of nitrogen/air to burn out the Styropor and to sinter the copper and the alloy particles together.

The hollow spheres are then activated in a 20 wt. % sodium hydroxide solution at 80° C. for 1.5 hours. The resulting activated hollow spheres have an average diameter of 6 mm, a jacket thickness in the range from 600 to 700 μμ and a bulk density of 0.60 g/ml. As can be seen visually from the evolution of hydrogen bubbles, the catalyst has a large reservoir of active hydrogen.

### EXAMPLE 8

A coating solution is prepared by suspending 800 g of an alloy of 50% Cu/50% Al and 104 g copper powder in 1,000 ml aqueous solution with a content of 5 wt. % polyvinyl alcohol and 1.25 wt. % glycerol. This suspension is then sprayed on to 2,000 ml polystyrene beads in the range from 4 to 5 mm, while these are suspended in upwards-flowing air. After the polystyrene beads have been coated with the abovementioned solution, the beads are dried in upwards-flowing air at temperatures of up to 80° C. Higher temperatures can also be used. These dried, coated polystyrene beads have a bulk density of 0.26 g/ml, and half of these beads are coated further with an alloy solution.

The solution for the second layer comprises 800 g of an alloy of 50% Cu/50% Al and 104 g copper powder suspended in 1,000 ml aqueous solution with a content of 5 wt. % polyvinyl alcohol and 1.25 wt. % glycerol. This suspension is then sprayed on to 1,000 ml of the abovementioned polystyrene beads which have been precoated with Cu/Al and dried, while this is suspended in an upwards-directed stream of air.

After the polystyrene beads have been coated with the abovementioned solution, the beads are dried in upwards-flowing air at temperatures of up to 80° C. Higher temperatures can also be used. The dried, coated beads are then heated at 550° C. in a controlled stream of nitrogen/air to burn out the Styropor and to sinter the copper and the alloy particles together.

The hollow spheres are then activated in a 20 wt. % sodium hydroxide solution at 80° C. for 1.5 hours. The resulting activated hollow spheres have an average diameter of 6 mm, a jacket thickness in the range from 600 to 700 μμ and a bulk density of 0.60 g/ml. As can be seen visually from the evolution of hydrogen bubbles, the catalyst has a large reservoir of active hydrogen. Hexachloroplatinum is then added to the suspension of the washed catalyst. The pH is adjusted and the suspension is stirred further. The doped catalyst is then washed. The platinum content of the catalyst is 1%.

### EXAMPLE 9

A coating solution is prepared by suspending 800 g of an alloy of 50% Cu/50% Al and 104 g copper powder in 1,000 ml aqueous solution with a content of 5 wt. % polyvinyl alcohol and 1.25 wt. % glycerol. This suspension is then sprayed on to 2,000 ml polystyrene beads in the range from 4 to 5 mm, while these are suspended in upwards-flowing air. After the polystyrene beads have been coated with the abovementioned solution, the beads are dried in upwards-flowing air at temperatures of up to 80° C. (Higher temperatures can also be used). These dried, coated polystyrene beads have a bulk density of 0.26 g/ml, and half of these beads are coated further with an alloy solution.

The solution for the second layer comprises 800 g of an alloy of 50% Cu/50% Al and 104 g copper powder suspended in 1,000 ml aqueous solution with a content of 5 wt. % polyvinyl alcohol and 1.25 wt. % glycerol. This suspension is then sprayed on to 1,000 ml of the abovementioned polystyrene beads which have been precoated with Cu/Al and dried, while these are suspended in an upwards-directed stream of air.

After the polystyrene beads have been coated with the abovementioned solution, the beads are dried in upwards-flowing air at temperatures of up to 80° C. Higher temperatures can also be used. The dried, coated beads are then heated at 550° C. in a controlled stream of nitrogen/air to burn out the Styropor and to sinter the copper and the alloy particles together.

The hollow spheres are then activated in a 20 wt. % sodium hydroxide solution at 80° C. for 1.5 hours. The resulting activated hollow spheres have an average diameter of 6 mm, a jacket thickness in the range from 600 to 700 μμ and a bulk density of 0.60 g/ml. As can be seen visually from the evolution of hydrogen bubbles, the catalyst has a large reservoir of active hydrogen. Iron(III) chloride is then added to the suspension of the washed catalyst. The pH is adjusted and the suspension is stirred further. The doped catalyst is then washed. The iron content of the catalyst is 3%.

### EXAMPLE 10

Preparation of Iminodiacetic Acid with a Fixed Bed Raney Copper Catalyst.

The example illustrates the conversion of diethanolamine (DEA) into the sodium salt of iminodiacetic acid (IDA) with the fixed bed Raney copper catalysts.

The experiments are carried out in a fixed bed tubular reactor with a liquid circulation. The following batch is initially introduced into the fixed bed tubular reactor:

100-400 g diethanolamine (3 mol)

266-1064 g aqueous NaOH solution (30 wt.-%). The ratio to diethanolamine is 2.66

200 g fixed bed Raney copper catalysts according to the invention

186-744 g H₂O, degassed with ultrasound. The ratio to diethanolamine is 1.86

The fixed bed tubular reactor is forced to a pressure of 10 bar with nitrogen and brought to the reaction temperature (TR=170° C.). After the reaction has started, the hydrogen formed is let off, the amount released being determined via a dry gas meter. The reaction is interrupted after a duration of 5 h and the autoclave is cooled. During the reaction, samples of the reaction solution are taken and are analysed by separation by gas chromatography.

The catalyst employed can be recycled several times without a noticeable loss of activity.

Further variations and modifications of the foregoing will be apparent to those skilled in the art and are intended to be encompassed by the claims appended hereto.

German priority application 00103547.6 is relied on and incorporated herein by reference.

We claim:

1. A fixed bed Raney copper catalyst, in the form of a tablet, extrudate, hollow body, fiber tablet, granule or disc-shaped granule, optionally bonded to a support.

2. The fixed bed Raney copper catalyst as claimed in claim 1, which is doped with one or more metals selected from the group consisting of iron, a noble metal, and mixtures thereof.

3. The fixed bed Raney copper catalyst as claimed 2, wherein claim 2, wherein the doping metal selected is alloyed into the copper.

4. The fixed bed Raney copper catalyst as claimed in claim 2, wherein the doping metal is subsequently coated on to the copper.

5. The fixed bed Raney copper catalyst as claimed in claim 2, which additionally comprises at least one other doping metal.

6. The fixed bed Raney copper catalyst according to claim 2 which additionally contains a member selected from the group consisting of Bi, Sn, Sb, Pb, Ge, Cr, Mo, Ti, Ni, Ta, Zr, V, Mn, W, Co, Nb and mixtures thereof.

7. The fixed bed Raney copper catalyst according to claim 2 which contains a doping element in the amount of 10 ppm to 1 wt. %.

8. The fixed bed Raney copper catalyst according to claim 2 wherein the noble metal is present in the amount of 10 to 50,000 ppm.

9. The fixed bed Raney copper catalyst according to claim 1 which has an average particle size of 0.05 mm to 20 mm.

10. A process for the preparation of the fixed bed Raney copper catalyst as claimed in claim 1, which comprises preparing a fixed bed Raney copper catalyst by the known route, shaping it, activating it, doping it with at least one doping metal, washing it and drying it.

11. A process for the dehydrogenation of an alcohol comprising contacting said alcohol at elevated temperature with the catalyst according to claim 1 and releasing hydrogen.

12. The process according to claim 11 wherein the alcohol is in the form of an aqueous alkaline solution.

13. The process according to claim 12 wherein the alcohol is under elevated pressure.

14. The process according to claim 11 wherein the alcohol is an amino alcohol or glycol.

15. The process according to claim 10 further comprising shaping the Raney copper catalyst into a hollow sphere by spraying an alloy powder of Raney copper alloy onto combustible beads, burning out the combustible beads to obtain hollow spheres and activity said spheres by contacting with sodium hydroxide solution and doping by applying a metal salt solution, washing and drying.
